(19) United States
(12) Patent Application Publication
(10) Pub. No.: US 2020/0025735 A1
(43) Pub. Date: Jan. 23, 2020

(54) GAS MULTIPLE MEASURE MODULE AND MEASURE PROCESS

(71) Applicant: Giovanni Mario Bardoni, Bognanco San Lorenzo (VB) (IT)

(72) Inventor: Giovanni Mario Bardoni, Bognanco San Lorenzo (VB) (IT)

(21) Appl. No.: 16/484,351

(22) PCT Filed: Feb. 14, 2018
(86) PCT No.: PCT/IT2018/050020

§ 371 Date: Aug. 7, 2019

(30) Foreign Application Priority Data
Feb. 22, 2017 (IT) 102017000020189

(51) Int. Cl. G01N 33/00; G08B 21/18; G08B 7/06

(52) U.S. Cl. CPC G01N 33/0031; G01N 33/0063; G01N 33/0057; G08B 7/06; G01N 33/0029; G08B 21/182; G01N 33/0022

(57) ABSTRACT

A system of gas multiple measurement having independent channels, wherein the gas measurement is carried out by a system comprising individual or redundant sensors configured to take contemporaneous double redundancy measures, such as contemporaneous measurement of flammable and/or toxic gases, with detection of various gases which, when present in the air and not detected, may cause the formation of explosive and/or toxic mixtures.

The present invention relates to the field of measurement of combustible, flammable and toxic gases present in air, more particularly caused by leakages of pipes, evaporation and similar leaks of potentially toxic and/or explosive gases. One of the sectors of mail use of the present invention is the extraction, production, treatment and conversion of petrochemical products in liquid and/or gaseous phase which may evaporate in air, so that gas environmental measurement is very important, mainly to protect and safeguard the operative personnel for instance of drilling, extraction, transport and stocking plants. Examples of risky environments surely are oil and gas treatment places such as oil wells and platforms and gas processing plants with high risk of presence of toxic and/or flammable gases generated by various industrial treatments. Therefore, environments of interest for the present invention may be sea and ground drilling and extraction plants such as oil wells, chemical and petrochemical plants, industries converting and stocking flammable gases, liquid and gaseous hydrocarbons, industries converting the raw materials into other fuels like gasoline, gas oil, hydrogen, acetylene, ammonia and other derivatives such as fertilizers, fats, lubricants and so forth.

The measurement devices in the field of the invention are also useful to monitor converted products like fuels containing toxic gases also in the liquid phase, which may release combustible and/or toxic gases even at room temperature. The gases, whose measurement will be the object of the present invention, when released in the atmosphere may endanger life of workmen and cause blasts and fires in spite of steady checks and safety measures such as compulsory alarms and protections, which unfortunately proved frequently to be inefficient or inadequate.

These gas measurement systems belong to those technically identified as data acquisition systems for personal and device safety systems. The data acquisition chain or channel has the function of storing information gathered from a physical transformation such as evaporation of liquid, semisolid and solid substances containing concentrations in air of one or more gaseous combustible and/or toxic substances. This information is converted into an analogic electric signal and the latter after a signal digitization process is stored in a microprocessor and put at disposal of the user.

The sensitive elements in the sensor body comprising transducer and their casing, are the first elements of the data acquisition chain or channel. Their function is to acquire as input a physical quantity and to supply as output an electrical quantity. This portion of the chain may comprise several parts, not all of them being always present, depending upon the specific task assigned to the circuit. They should generate the sensor output signal so as to fall within the parameters of the analog-digital converter thus obtaining a signal that does not generate any kind of error. This requires the presence of gas measuring sensor having various sensitive element, in order to obtain the contemporaneous sure measurement of different gases at the same point.

Task of the converter is to transform the analogic signal at its input into a digital signal. Since the analogic quantities are continuously varying and may assume infinite values within a given interval, a quantity must correspond to the analogic signal, which can assume a finite set of values, mutually separated by a constant interval called quantum corresponding to one-bit value. At last the microprocessor is the main element managing the acquisition chain together with the data distribution and transmission chain. Its task is to acquire data converted by the sensitive element and adapt said data for the transmission.

The present invention concerns innovative systems for detecting toxic or explosive gases, comprising specific devices to detect such gases in a redundant mode to ensure their measurement and the consequent protection of operators and investors. The measure module and more particularly the basic measurement chain proposed by the present invention were carried out to solve the well-known serious problems of the prior art detection devices which could not prevent a number of poisoning of operators and even the full destruction of sea platforms and extraction and conversion ground plants, that were all considered theoretically protected.

The systems for detecting combustible and toxic gases for fixed installation available on the market generally comprise only one type of gas sensor using only one measurement technology. Each sensor of this kind is usually connected to one single electronic transmitter. More particularly, this single channel measurement type may possibly require the installation of various detectors. However, when there is one single detector for one single gas, the possible problems may be even more serious. Indeed, two or more gases are present in a certain area, and the detectors are not properly positioned or two different types of detectors are not available, this circumstance involving the lack of contemporaneous detection of said two gases in the same area, in case of escape of one or more toxic and flammable gases, then toxic and explosive mixtures might be generated if both gases are not quickly detected, and consequently the results could be not only dangerous, but even devastating for the operators, the area, the production plant and the investment. As above mentioned, unfortunately there is a long history of operator poisonings, explosions of factories, sea platforms and ground plants.

A technical solution solving many problems of the prior art systems, more particularly those connected with the presence of a single measurement channel, is the solution proposed in Italian Patent Application MI2014A001484 of the same Applicant (now patent IT 1425552 and also published as WO 2016/030735 A1) that introduced in these systems the possibility of operation with two independent channels, thus allowing to make discrete measurements of two different toxic and explosive gases or redundant measurements of the same gas, but in any case not at the same time. However, it is to be noted that this technical solution cannot be used to carry out redundant measurements of two different gases in air dispersed gas mixtures comprising for instance flammable gases and toxic gases that can be detected with two different technologies of sensors.

Moreover, the largely used present sensors available on the market, for instance like the type described in the above cited patent literature, require that both the transmitter and the sensor are provided with their own separated and dedicated electronics, and consequently connection and linking elements between the two devices, this requirement involving high structural and installation costs of the module.

An object of the present invention is the provision of a measure module solving the above-mentioned problems.

More particularly, an object of the present invention is the provision of a measure module allowing to carry out the contemporaneous detection of four measurements of one or two different types of gases even with double redundancy at one single measure point.

Another object of the present invention is the provision of a module where the sensor and transmitter elements are arranged in a single casing and the manufacturing costs are reduced, limiting the electronic and mechanical parts of the module.

Still an object of the present invention is to optimize the manufacturing and handling costs for the user.

An essential object of the present invention is the improvement of safety of persons and installations by means of a warranted measurement of the danger through a redundant detection of toxic and flammable gases in all the hereinbefore cited fields and other sectors where said module may result as needed to increase safety.

It is also an object of the present invention to illustrate a measure module which is performing, reliable, simple to be installed and used, as well as easy to be maintained and serviced efficiently.

A further object of the present invention is the provision of a measuring process which is very reliable when compared to prior art systems and improves the safety standards of personnel and location where the measure instrument is installed, even considering possible signal loss along the measure channel.

At last, another object of the present invention is the provision of such a module configuration that the required filter is not part of the sensor, but it can be easily removed to be individually cleaned or replaced in few minutes without requiring full change of the sensor, which is a decidedly expensive operation that lasts some hours in the traditional systems available on the market.

These and other objects will be attained by the gas multiple measure module according to the present invention, which is provided with multiple channels as hereinafter described, wherein the gas detection is carried out by a module comprising redundant sensors configured to make contemporaneous measurements with double redundancy, such as contemporaneous measurement of flammable and/or toxic gases, with detection of various gases which, when present at the same time, may generate very dangerous explosive and/or toxic mixtures.

Moreover, this measure module allows to detect four independent measurements at the same point, and this allows to optimize the installation costs, to improve measurement reliability and to simplify the checking and servicing operations.

Another advantage of the present invention is the provision of a very compact detection module having a limited number of electronic and mechanical components.

Finally, an advantage of the present invention is the provision of a measure module allowing the transmission of advanced information, still keeping a simple and analogic communication cabling.

More particularly this aspect, jointly with the other above-mentioned advantages and others to be described hereinafter, considerably increases safety of the work environment together with the plant protection, which are the objects of the present invention.

The subject of the present invention will be illustrated in detail by the accompanying drawings and their relevant description, wherein only some of the possible innovative embodiment will be discussed, in which:

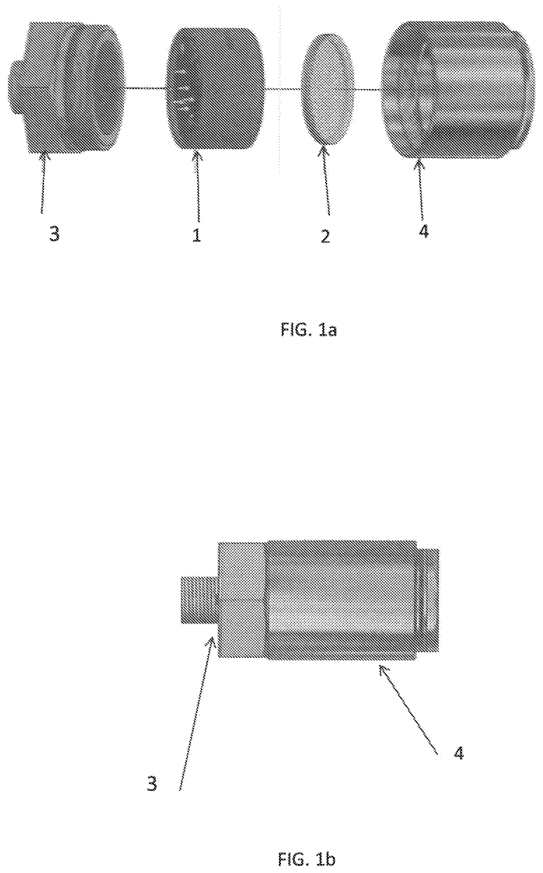

FIG. 1a is an exploded view of an embodiment of the gas multiple measure module of the invention;

FIG. 1b is a view of an example of embodiment of the assembled module of gas multiple measurement of the invention;

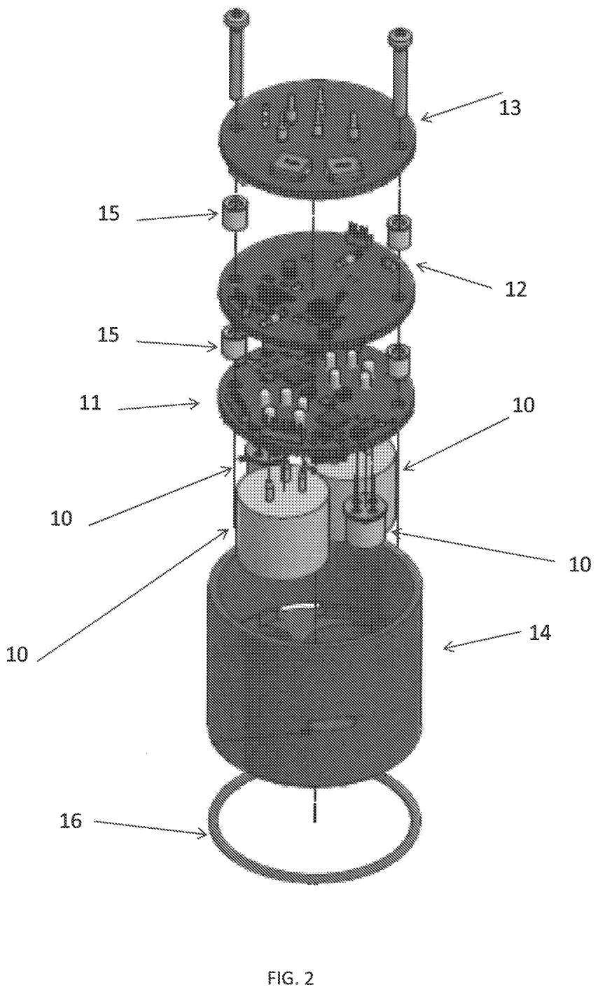

FIG. 2 is an exploded view of an example of embodiment of the sensor-transmitter housing 1 of the gas multiple measure module; and

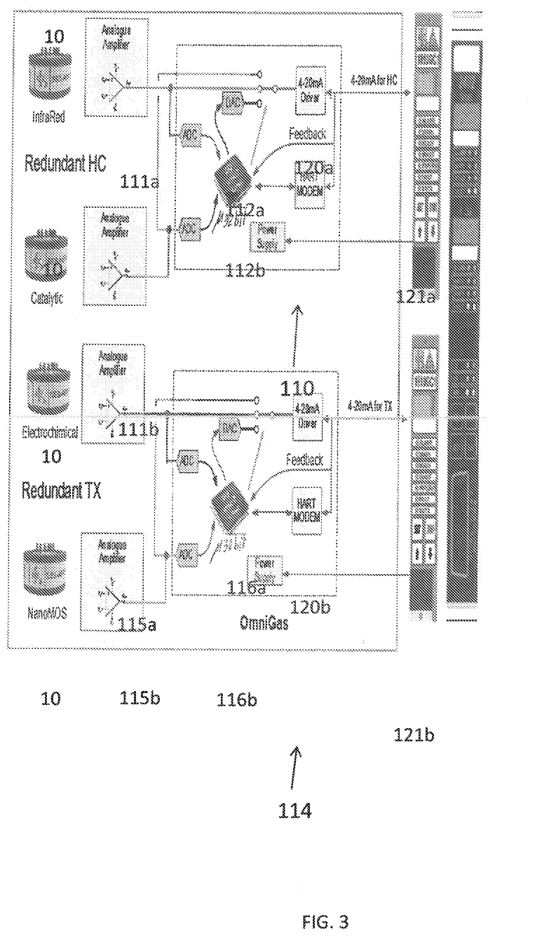

FIG. 3 shows the operative diagram of the embodiment of the sensor-transmitter housing 1 of FIG. 2.

With reference now to FIGS. 1a and 1b, the main components of the gas multiple measure module of the invention are shown, namely the module head 3, the sensor-transmitter housing 1, the filter 2 and the cover casing 4 enclosing the sensor-transmitter housing 1 and the filter 2 and connected with the module head 3 thus forming the module of the invention. This module of the invention will be part of a more complex system comprising a power supply, at least a monitor to display the gas measurement, a luminous signalling system such as a gas alarm blinker, including at least an audible warning device such as a gas alarm siren, all mounted on a column or similar support.

A particular feature of the module of the invention is that the filter 2 (for instance a sintered element) is inserted in the module cover casing 4 and may be easily released therefrom, so that said filter 2 may be easily cleaned or removed when it has to be replaced or the sensors 10 should be changed.

The sensor-transmitter housing 1 comprises a socket 14 adapted to contain, enclose in proper templates and protect the other components of the sensor-transmitter housing 1, namely an output connection board 13, an input PCB board 12 and a sensor PCB board 11 to which up to four sensors 10 are connected, thus the elements allowing to detect the specific toxic, explosive or polluting substance present in the air, where said sensors 10 may be installed in the sensor-transmitter housing 1 in several configurations, namely a pair of sensors 10 (first and second sensor 10) of one type for measure of toxic gas (for example nMos+nMos), and a different pair of sensors 10 (third and fourth sensor 10) of another type for measure of explosive gas (for example Ir+Ir), so as to measure by double redundancy with reliable members two substances of different nature (a toxic one and an explosive one); or a pair of sensors 10 of the same kind and two additional sensors 10 of different technology but adapted to detect the sane substance even in presence of inhibiting substances critical for one of the two technologies; or at last four sensors 10 all different from the others so as to detect two substances in redundant mode but with four different technologies thus ensuring the maximal certainty of the measurement in case of presence of inhibiting substances.

As above discussed, the sensor-transmitter housing 1 comprises a sensor PCB board 11 provided with analog amplifiers 111, 115 specific for each sensor 10, required to amplify the signal from each sensor 10. Connected downstream each first analog amplifier 111a, 111b and second analog amplifier 115a, 115b, first analog-digital converters 112a,112b and second analog-digital converters 116a, 116b are provided, which are necessary to convert the analog signal coming from the corresponding amplifier, that is from the corresponding sensor, into a digital signal to be used by the corresponding first controller 110 and second controller 114 both at 32 bits or higher. The described sensor PCB board 11 in addition comprises at least a pair of digital-analog converters 120 each output connected, thus downstream each controller 110, 114 of the sensor PCB board 11 to convert the signal from digital to analog.

The module of the present invention therefore is provided with two independent measure channels: a first measure channel comprises a first sensor 10, a second sensor 10, first analog amplifiers 111a and 111b, a pair of first analog-digital converters 112a and 112b, a first controller 110, a first digital-analog converter 120a, a first output HART 121a and the power supply of the measure channel; a second measure channel comprises a third sensor 10, a fourth sensor 10, second analog amplifiers 115a and 115b, a pair of second analog-digital converters 116a and 116b, a second controller 114, a second digital-analog converter 120b, a second output HART 121b and the power supply of the measure channel.

According to the present invention the measurement process occurs in the following way: when for instance the first controller 110 receives redundant data from the two relevant sensors 10 connected upstream it, the controller compares the data computing the difference between the two detected instant values, e.g. X1, X2, and transmits as output, possibly also to a display (not shown), if the difference is lower than a predetermined threshold value, only the higher data of the two values, e.g. X1, this being in favour of the safety target. However, if the value of the difference between X1 and X2 is higher than the threshold, then the controller transmits as output an alarm signal.

Now in the illustrated example, the controller may also verify if the value of current intensity associated with the measure X1 is congruent with the X1 data; in other words, as to each value that the variable X1 may assume, corresponds a limited range of possible values of intensity of output current (indeed the sensor translates the concentration of the detected substance into an electric pulse of different intensity according to the value of concentration of the detected substance) and this value of current intensity may undergo electric interference because of a number of various grounds and not only in case of the sensor malfunction, the controller verifies if the value of current intensity generated by the value of the X1 measure is congruent with the expected value of current intensity related to such a value of X1 concentration, and in the affirmative case the X1 value is approved and confirmed as output, otherwise an alarm signal is being transmitted which can be seen by the operator, who must then contact the service to verify the correct operation of the gas multiple measure module.

Therefore, the measure process implemented in the gas multiple measure module of this invention for instance through the first measure channel comprises the following steps:

Measuring concentration X1 of the substance X by a first sensor 10 and at the same time measuring concentration X2 of substance X by a second sensor 10;

Comparing the two values X1 and X2 of substance X through a first controller 110 and calculating the difference d=X1−X2;

If the difference d is lower than a predetermined threshold S, transmitting the higher value between X1 and X2, for instance X1;

If said difference is greater than the predetermined threshold S, transmitting as output an alarm signal through a display or other visual or acoustic signalling to an operator.

Moreover, both the first and the second measure channel may also implement the following steps in the measure process:

If the current intensity corresponding to the detected value X1 is not congruent, within a predetermined tolerance, with the current intensity value expected for such a detected value X1, the first controller 110 sends a proper alarm signal through a display or other visual or acoustic signalling to an operator.

This process may be implemented contemporaneously in the same way and following the same steps, also in the second measure channel for detection of the substance Y, and in this case the variable Y will be substituted for the variable X.

Moreover, the supply PCB board 12 is operatively connected with the sensor PCB board 11, which is provided with at least a pair of analogic connections coming from the first controller 110 and the second controller 114, at least two pairs of analogic outputs directly coming from the first analogic amplifiers 111 and the second analogic amplifiers 115 associated to sensors 10, as well as a pair of analogic outputs HART 121, also configured to receive outside data from additional devices that may be connected to said module, and two power supplies, that is one for each measure channel.

An output connection board 13 is operatively connected with the supply PCB board 12, and in its turn comprises EMC and ESD components, to warrant the electromagnetic compatibility and the protection against electrostatic discharges of the sensor-transmitter body group 1.

The sensor PCB board 11, the supply PCB board 12 and the output connection board 13 are arranged mutually spaced through suitable spacers 15 and fixed to each other by conventional fastening means.

Some of the possible configurations of sensors 10 may be as follows:

a) IR+IR−nMos+nMos (being able to implement double redundancy measures of combustible gases by infrared technology and toxic gases by nMos technology);

b) Cat+Cat−nMos+nMos (being able to implement double redundancy measures of combustible gases by catalytic elements and toxic gases by nMos or nanoMos elements);

c) Cat+Cat−ELC+ELC (being able to implement double redundancy measures of combustible gases by catalytic elements and toxic gases by elements sensitive to electrochemical technology; d) nMos+nMos−ELC+ELC (being able to detect combustible gases by nMos technology and toxic gases by electrochemical technology);

e) IR+Cat+nMos+ELC, wherein IR is used for measuring hydrocarbon gases, Cat for hydrogen gas (% H2), nMos for measuring PPM (parts per million) of H2, and ELC for measuring PPM pf toxic gases (such as H2S, SO2, HCl, CL2, NO2 and so forth).

Therefore, the module of the present invention may be so configured, that the first sensor 10 is of same technology of the second sensor 10, and/or the third sensor 10 is of the same technology of the fourth sensor 10, or the four sensors 10 are all of different technology.

It has to be noted that each element with a specific name or a specific quantity of data transmission is herein indicated as an example of particularly preferred embodiments of the innovative gas detection module herein described, as the sensor-transmitter body containing up to four sensors 10 in pairs or of individual different technology has to carry out a specific function which is being programmed for an application requested by the used and stored as such in the reference microprocessor.

The above described sensor-transmitter body 1 is connected through Protocol HART 121 wired or wireless or through other connection systems, to remote control devices or to a display in such a way that the operator may easily see the amount of the detected measurement values, and if required take accordingly proper actions in view of the relevant connection.

The sealing ring 16 is applied to the assembled sensor-transmitter body on the side where the sensors 10 are disposed and its purpose is to prevent inlet of air, humidity, water, powders or other substances inside the sensor-transmitter body 1.

To the above described module for gas multiple detection of the present invention, sound and light operated signalling systems may also be connected, which are actuated, when instantaneous values are measured which are exceeding a predetermined threshold, through common control boards receiving analogic signals 4-20 mA, as well as on site or remote monitoring and managing systems of continuously detected data.

In a particularly advantageous way, the sensor-transmitter body 1 is configured in such a way, that the entire electronic portion is integrated in the sensors or the sensitive elements, without requiring additional electronic components or junctions between sensors and transducers, if they were located on different elements.

Use of gas detectors with various integrated sensors and transmitters is the optimal solution for the total protection of gas and oil extraction platforms and plants constituting high risk environments, ensuring a higher reliability for operators and investments and at the same time a scale economy, thanks to the realization of an integrated module of various single or paired sensitive elements into one single sensor of multiple measurement of different gases, formed by the installation column of the components of the measurement, alarm and warning module, all included in a single solution. These features are indispensable conditions in oil & gas plants, refineries, chemical and petrolchemical factories which are high risk installations where the joint presence of toxic and explosive gases and the consequent fire danger is very high.

Additional variations in which the module elements are differently arranged by opposition, engagement, junction or alternative assembling features of the module elements, such as different embodiments of the transducers or alternative positions of one or more sensors, as well as variations to the detection module in the parts not strictly concerning the innovative module described in the present invention, should be considered object of the present invention, in which therefore even modifications and changes concerning for instance the geometries chosen for the various movable and fixed elements, the materials adopted for each member, and also the specifications of the actuation module, or modifications to software adaptation, may be made however without departing from the scope of protection of the present invention as defined in the appended claims.

### LIST OF REFERENCE NUMERALS

1 sensor-transmitter body

10 sensors

11 sensor PCB board

110 first controller

111a, 111b first analogic amplifiers

112a, 112b first analog-digital converters

114 second controller

115a, 115b second analogic amplifiers

116a, 116b second analog-digital converters

120a first digital-analog converter

120b second digital-analog converter

12 power supply PCB board

121a first HART output

121b second HART output

13 output connection board

14 housing body

15 spacers

16 sealing ring

2 filter

3 module head

4 cover casing

1. A module for multiple gas measuring comprising:
a module head,
a sensor-transmitter body,
a filter, and
a cover body enclosing the sensor-transmitter body and the filter, wherein the cover body is connected to the module head for assembling said module,
wherein said sensor-transmitter body comprising:
a first measure channel comprising a first controller, a first sensor, and a second sensor for measuring substance X, and
a second measure channel comprising a second controller, a third sensor, and a fourth sensor for measuring substance Y,
wherein said first and second measure channels being independent mutually, said measure channels being configured for allowing redundant measures of two substances present in air to be measured, and
wherein furthermore the filter is configured to be easily cleaned and disassembled when it is necessary to replace it or the sensors.

2. The module according to claim 1, wherein the sensor-transmitter body further comprises a sensor printed circuit board (PCB) to which are connected the four sensors, said sensor PCB having first analog amplifiers and second analog amplifiers specific for each of said sensors, wherein connected downstream from each of said first analog amplifiers and second analog amplifiers are arranged first analog-digital converters and second analog-digital converters, for converting an analogic signal into a digital signal usable by the corresponding first controller and second controller, said module further comprising first and second digital-analog converters connected downstream from the first and second controllers, respectively, of sensor PCB, for converting the controller signals from digital to analog.

3. The module according to claim 1, wherein both the first controller and the second controller are 32 bit controllers or higher.

4. The module according to claim 1 comprising a power PCB functionally connected to sensor PCB, said power PCB comprising power supplies for each measure channel and two analog highway addressable remote transducer (HART) outputs configured also for receiving data from further devices connectable to said module.

5. The module according to claims 4 wherein the power PCB is provided with at least two analog connections, coming from the first controller and the second controller, at least two pairs of analog outputs coming directly from first analog amplifiers and second analog amplifiers associated with the sensors.

6. The module according to claim 1 further comprising:
an output connection PCB connected to the power PCB, said output connection PCB comprising in turn electromagnetic compatibility (EMC) and electrostatic discharge (ESD) components, in order to ensure electromagnetic compatibility and protection against electrostatic discharge of the sensor-transmitter body and
suitable spacers for electrically isolating the sensor PCB, the power PCB and the output connection PCB, which are fastened to each other through known fastening means, said module comprising further a seal arranged on the sensor-transmitter body on the side where the sensors are positioned, once the measure module is assembled.

7. The module according to claim 1, wherein the first sensor is of the same technology as the second sensor (10) and/or the third sensor is of the same technology as the fourth sensor.

8. A measuring process being implemented in a first measure channel of a measure module for multiple gas measuring comprising the following steps:
measuring concentration X1 of a substance X through a first sensor and at the same time measuring concentration X2 of the substance X through a second sensor,
comparing the two values X1 and X2 of the substance X through a first controller and computing the difference, d=X1−X2,
wherein if said difference d is lower than a predetermined threshold S, sending the higher value between X1 and X2, for instance X1, or
if said difference d is higher than the predetermined threshold S, sending an alarm output signal through a display or visual or acoustic communication to the operator.

9. The measuring process according to claim 8 further comprising the following step:
if current intensity corresponding to value X1 measured is not congruent, within a predetermined tolerance, with the expected value of current intensity corresponding to said measured X1 value, the first controller sends an alarm signal through a display or visual or acoustic communication to the operator.

10. The measuring process according to claim 8 wherein the process is implemented at the same time, in the same manner and following the same process steps also in the second measure channel for measuring a substance Y.
